(19) United States
(12) Patent Application Publication
(10) Pub. No.: US 2005/0118125 A1
(43) Pub. Date: Jun. 2, 2005

(54) Underarm products with superabsorbent component

(75) Inventors: Jairajh Mattai, Piscataway, NJ (US); Suman Chopra, Dayton, NJ (US); Peter Hilliard JR., Far Hills, NJ (US); Eric Guenin, Pennington, NJ (US); Ronald Jacoby, Holland, PA (US); Elizabeth Linn, Lyndhurst, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 10/964,268

(22) Filed: Oct. 13, 2004

Related U.S. Application Data
(63) Continuation-in-part of application No. 10/696,764, filed on Oct. 29, 2003.

(51) Int. Cl. A61K 7/32

(52) U.S. Cl. 424/65

(57) ABSTRACT

A stick or soft solid suspension product comprising: (a) 0.01-20 weight % of a polyacrylate superabsorbent polymer (sodium salt), with a salt or ionic strength tolerance under a Baseline Absorption Test sufficient to give at least 25 weight % water absorption; (b) 10-88 weight % of a volatile silicone having a flash point of 100 degrees C. or less; (c) a selected gelling agent; provided that the water content is ≦2 weight % and the ratio of superabsorber to active salt is in the range of 0.13-4:1.

### FIELD OF THE INVENTION

This invention relates to suspension products that are useful to reduce underarm wetness, for example, antiperspirant and/or deodorant agents. These products are particularly advantageous in providing deodorants that have reduced wetness without the use of an antiperspirant active. They are also advantageous in providing antiperspirants with additional wetness benefits. This application is a continuation-in-part of copending case U.S. Ser. No. 10/696,764, filed Oct. 29, 2003.

### BACKGROUND OF THE INVENTION

A variety of technologies have attempted to use superabsorbent polymers of various types in a wide variety of applications. These technologies include the construction of diaper products for children and adults, and the use of superabsorbent polymers to clean up liquid spills. The problems associated with the use of such polymers in personal care applications include a wet and sticky feel and skin irritation. Additionally, it has been difficult to find a way of applying such products in the underarm area in a way that results in an aesthetically acceptable product form.

It has now been found that selected superabsorbent polymers in certain formulations both with and without antiperspirant or deodorant agents may be used to create superior anti-wetness products. Because of the characteristic that they have some salt tolerant behavior, these polymers can also be used in the presence of antiperspirants to create superior wetness control.

### BRIEF SUMMARY OF THE INVENTION

The invention comprises an underarm product suitable for use to reduce wetness under the arm. It may be viewed as providing some deodorancy effect. Additionally, an antiperspirant active may be included to provide an antiperspirant/deodorant. This underarm product is a suspension product which may be a stick or soft solid and which comprises a superabsorbent polymer which is a surface modified sodium polyacrylate salt and which has a critical level of salt tolerance. The surface modification allows for greater water absorption in the presence of salt, i.e. ionic strength. While these homopolymers may be used in a variety of particle sizes, it is generally believed that the smaller sizes are preferred (for example, less than 100 microns, such as particle size of 20-30 microns, or, for certain types of superabsorbents, particle sizes of 200-300 microns can be tolerated).

The formulations of the invention may be made as antiperspirants and/or deodorants. In the case of antiperspirants, the products give an extra measure of protection against wetness. In the case of deodorants, the products may be made with low levels of antiperspirant active or with other agents which provide a deodorizing effect but which are not antiperspirant salts.

### DETAILED DESCRIPTION OF THE INVENTION

Products formulated according to the invention comprise suspension products which are sticks or soft solids comprising:
(a) 0.01-20 weight % (particularly 1-20%, especially 0.1-10% and, more particularly, 0.5-5%) of a polyacrylate superabsorbent polymer (sodium salt), with a salt or ionic strength tolerance under the Baseline Absorption Test described below sufficient to give at least 25 weight % water absorption (for example, materials having a mean particle size less than 100 microns);
(b) 10-88 weight % of a volatile silicone having a flash point of 100 degrees C. or less (particularly a D4-D6 cyclomethicone; and especially a D5 or D6 cyclomethicone or a combination of D5 and D6 cyclomethicones);
(c) a gelling agent selected from the group consisting of 5-30 weight % stearyl alcohol; 0.1-20 weight % waxes (for example, Japan wax, hydrogenated castor oil); 0.1-10 weight % (on an actives basis) silicone elastomer; 0.1-3 weight % siliconized polyamides; 0.1-20 weight % low molecular weight polyethylene having a molecular weight in the range of 400-1000 (for example 400 such as Performalene-400 from Baker Petrolite, Polymer Division, Sugar Land, Tex.) and combinations of the foregoing;
(d) 0-5 weight % of a surfactant with a hydrophilic/lipophilic balance (“HLB value”) in the range of 3-13 (for example, from 0.05-50 weight % (particularly 1-30%) of a silicone copolyol which is 10% in cyclomethicone, or its equivalent may be used for a soft solid);
(e) an antiperspirant or a deodorant ingredient such as 0-25 weight % (for example, 0.1-5 weight % if the antiperspirant active is used for deodorancy and not wetness control, 8-25% of an antiperspirant active if more wetness control is desired) or an effective amount of a deodorizing agent which is not an antiperspirant active;
(f) 0-20 weight % (particularly 5-10%) of a nonvolatile silicone having a flash point greater than 100 degrees C.; and
(g) 0-20 weight % (particularly 2-12%) of an emollient (for example, a member selected from the group consisting of C12-15 alkyl benzoate, PPG-3-myristyl ether, and hydrogenated polyisobutene (Polyisobutene 250)); provided that:
the water content is ≦2 weight % (as based on added water and excluding any waters of hydration such as with the antiperspirant salt) and the ratio of superabsorber to active salt is in the range of 0.13-4:1, particularly in the range of 0.18-3:1.

While no water is recited as being added, up to 2 weight % water may be present because of the types of raw materials used.

With regard to the amount of volatile silicone used in the invention, 10-88 weight % is used for stick products and soft solids, with the degree of hardness being controlled-by the use of gelling agents.

Optionally, one or more other ingredients can be used such as fragrance, coloring agents, antibacterial agents, masking agents, additional surfactants (such as PEG-8 distearate) or fillers (for example, talc).

The stearyl alcohol used in this invention is preferably a straight chain material with no unsaturation.

Examples of superabsorber materials that work in this invention include HySorb™ 8100 and HySorb™ CL-15 (from BASF, N.C.), preferably ground to particle size not exceeding 100 microns; AQUAKEEP J-550 and AQUAKEEP 10SH-N (from Kobo Products, Inc., South Plainfield, N.J.) also ground to a particle size in the range of less than or equal to 100 microns. (Note that J-550 has mean particle size of 200-300 microns, and 10SH-N has a mean particle size of 20-30 microns.)

A reduced particle size for the various types of superabsorbents described is desired to reduce/eliminate gritty feel in the product. For example, the HySorb™ products are preferred to have a particle size not exceeding 100 microns because of reducing gritty feel and larger particles will not form a satisfactory product in processing (for example, settling issues). A particular range of particle sizes includes superabsorbents having at least 80-85 weight % of the particles with a size ≦75 microns, and another particular range is from 6-65 microns with no more than 10% of the particles having a size less than 6 microns. For many of these products, grinding of stock material is needed to achieve the desired particle sizes.

While the invention has been described in terms of selected superabsorbers and actives, especially in a selected ratio, it is to be noted that modifications in type and amount of superabsorber may be made to ensure that the water absorbency requirements are met. Thus, in the Tables below, it will be described how to balance these two ingredients to achieve the effect.

Volatile silicones and silicone surfactants are also used in the invention. By volatile silicone material is meant a material that has a flash point of 100 degrees C. or less at atmospheric pressure. Such volatile silicones include conventional cyclic and linear volatile silicones such as cyclomethicone (especially cyclopentasiloxane, also called “D5”), “hexamethyldisiloxane”, and low viscosity dimethicone (for example, Dow Corning® 200 fluid having a viscosity of 0.5-5 centistokes). Illustratively, and not by way of limitation, the volatile silicones are one or more members selected from the group consisting of cyclic polydimethylsiloxanes such as those represented by Formula I: 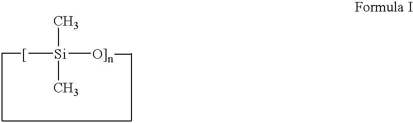
where n is an integer with a value of 3-7, particularly 5-6. For example, DC-245 fluid (or the DC-345 version) from Dow Corning Corporation (Midland, Mich.) is a type of cyclomethicone which can be used. These include a tetramer (or octylmethyl-cyclotetrasiloxane) and a pentamer (or decamethylcyclopentasiloxane). The volatile linear silicones can also be included in this group of volatile silicones and are one or more members selected from the group consisting of linear polydimethylsiloxanes such as those represented by Formula II: 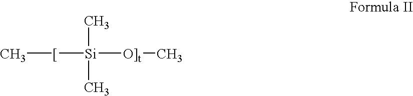
and t is selected to obtain a viscosity of 0.5-5 centistokes.

Examples of such volatile silicones include one or more members selected from the group consisting of D4, D5, and D6 cyclomethicones; and linear dimethicones having a viscosity in the range of 0.5-10 centistokes. Preferably the oil phase is a mixture of one or more of D4, D5 and D6 cyclomethicones.

Gelling agents include elastomers such as:
(a) a dimethicone/vinyldimethicone crosspolymer composition made by reacting (in the presence of a platinum catalyst) a polymethylhydrogensiloxane with an alpha, omega-divinylpolydimethyl siloxane for which the dimethicone/vinyldimethicone crosspolymer composition (1) is used at a concentration of 4-10% in cyclomethicone (particularly 4-7%, and, more particularly, 4-6.5%) (for example, where the cyclomethicone is a D4 or D5 cyclomethicone), (2) has a refractive index in the range of 1.392-1.402 at 25 degrees C., and (3) has a viscosity in the range of 0.013−1×10⁴ Pascal seconds; for example, one particular elastomer of interest is KSG-15 silicone elastomer from Shin-Etsu Silicones of America (Akron, Ohio).
(b) a cyclomethicone (and) dimethicone crosspolymer made with an ≡Si—H containing polysiloxane and an alpha, omega-diene of formula CH₂═CH(CH₂)ₓCH═CH₂, where x=1-20, to form a gel by crosslinking and addition of ≡Si—H across double bonds in the alpha, omega diene, which crosspolymer has a viscosity in the range of 50,000-3,000,000 centipoise (particularly 100,000-1,000,000; more particularly 250,000-450,000 centipoise; and most particularly 350,000 centipoise), preferably with a nonvolatiles content of 8-18% (particularly 10-14% and most particularly 12-13%) in cyclomethicone (for example a D4 or D5 cyclomethicone), (an example of such a crosspolymer composition being DC-9040 from Dow Corning Corporation (Midland, Mich.) with other types of such crosspolymers (also called elastomers) being described in U.S. Pat. No. 5,654,362, incorporated by reference herein as to the description of such polymers and methods of making such polymers);

Particular examples of suitable elastomers are SFE 167, a cetearyl dimethicone/vinyl dimethicone crosspolymer from GE Silicones (Waterford, N.Y.); SFE168, a cyclomethicone (and) dimethicone/vinyl dimethicone crosspolymer from GE Silicones; vinyl dimethicone crosspolymers such as those available from Shin-Etsu Silicones of America (Akron, Ohio) under trade names KSG-15 (cyclomethicone (and) dimethicone/vinyl dimethicone crosspolymer), KSG-16 (dimethicone (and) dimethicone/vinyl dimethicone crosspolymer), KSG-17 (cyclomethicone (and) dimethicone/vinyl dimethicone crosspolymer), KSG-18 (phenyl trimethicone (and) dimethicone/phenyl vinyl dimethicone crosspolymer); and KSG-20 (dimethicone copolyol crosspolymer; dimethicone/vinyl dimethicone crosspolymer from Dow Corning Corporation (Midland, Mich.) under trade name Dow Corning 9506 Cosmetic Powder, DC-9040 elastomer in cyclomethicone from Dow Corning; and a mixture of cyclomethicone and stearyl-vinyl/hydromethylsiloxane copolymer available from Grant Industries, Inc. (Elmwood Park, N.J.) under the trade name GRANSIL SR-CYC.

The gelling agent may include both high and low melting point waxes. An example of such a combination of waxes includes 5-23 percent stearyl alcohol and 2-5 percent hydrogenated castor oil (melting point in the range of 50-90 degrees C. such as about 80 degrees C.).

For gelling agents which are polyamides, one should include at least one siliconized polyamide of Formula IIIA: 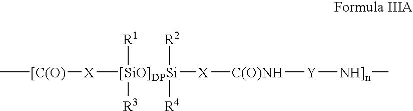
where:
(1) DP is a number in the range of 10-40 (particularly 15-30);
(2) n is a number selected from the group consisting of 1-500;
(3) X is a linear or branched chain alkylene having 1-30 carbons;
(4) Y is selected from the group consisting of linear and branched chain alkylenes having 1-40 carbons, wherein:
   (A) the alkylene group may optionally and additionally contain in the alkylene portion at least one of the members of a group consisting of (i) 1-3 amide linkages; (ii) C5 or C6 cycloalkane (as a cycloalkylene linkage); and (iii) phenylene optionally substituted by 1-3 members selected independently from the group consisting of C1-C3 alkyls; and
   (B) the alkylene group itself may optionally be substituted by at least one member selected from the group consisting of (i) hydroxy; (ii) C3-C8 cycloalkane; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or Y=Z², where 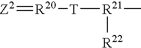
      wherein each of R²⁰, R²¹ and R²² are independently selected from the group consisting of linear and branched C1-C10 alkylenes; and T is selected from the group consisting of (i) a trivalent atom selected from N, P and Al; and (ii) —CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl, especially methyl and ethyl and most especially methyl; and
(5) each of R¹-R⁴ is independently selected from the group consisting of methyl, ethyl, propyl, isopropyl, a siloxane chain, and phenyl, wherein the phenyl may optionally be substituted by 1-3 members from the group consisting of methyl and ethyl (with more particular values for R¹-R⁴ being selected from methyl and ethyl and especially methyl);
   wherein the polyamide of Formula IIIA has:
   (i) a silicone portion in the acid side of the polyamide;
   (ii) a degree of polymerization in the range of 10-40 (particularly 15-30);
   (iii) an average molecular weight of at least 50,000 daltons (particularly in the range of 80,000-150,000 daltons and, more particularly in the range of 90,000-120,000 daltons) with at least 95% of the polyamide having a molecular weight greater than 10,000 daltons; and
   (iv) a polydispersity of less than 20 (particularly less than 4).

Suitable silicone surfactants include silicone polyglucosides (for example, octyl dimethicone ethoxy glucoside) and silicone copolyols having an HLB value (hydrophilic lipophilic balance) in the range of 3-13. A silicone copolyol (especially dimethicone copolyol) may be used in an amount of 0.05-5.0weight % (actives basis), particularly 0.1-3.0% and, more particularly, 0.1-2.0%.

In general, silicone copolyols useful in the present invention include copolyols of the following Formulae IV and V. Formula I materials may be represented by:
(R¹⁰)₃—SiO—[(R¹¹)₂—SiO]ₓ—[Si(R¹²)(R^{b}—O—(C₂H₄O)ₚ—(C₃H₆O)ₛ—R^{c})O]_{y}—Si—(R¹³)₃ Formula IV

wherein each of R¹⁰, R¹¹, R¹² and R¹³ may be the same or different and each is selected from the group consisting of C1-C6 alkyl; R^{b} is the radical —CₘH₂ₘ—; R^{c} is a terminating radical which can be hydrogen, an alkyl group of one to six carbon atoms, an ester group such as acyl, or an aryl group such as phenyl; m has a value of two to eight; p and s have values such that the oxyalkylene segment —(C₂H₄O)ₚ—(C₃H₆O)ₛ— has a molecular weight in the range of 200 to 5,000; the segment preferably having fifty to one hundred mole percent of oxyethylene units —(C₂H₄O)ₚ— and one to fifty mole percent of oxypropylene units —(C₃H₆O)ₛ—; x has a value of 8 to 400; and y has a value of 2 to 40. Preferably each of R¹⁰, R¹¹, R¹² and R¹³ is a methyl group; R^{c} is H; m is preferably three or four whereby the group R^{b} is most preferably the radical —(CH₂)₃—; and the values of p and s are such as to provide a molecular weight of the oxyalkylene segment —(C₂H₄O)ₚ—(C₃H₆O)ₛ— of between about 1,000 to 3,000. Most preferably p and s should each have a value of about 18 to 28.

A second siloxane polyether (copolyol) has the Formula V:
(R¹⁰)₃—SiO—[(R¹¹)₂—SiO]ₓ—[Si(R¹²)(R^{b}—O—(C₂H₄O)ₚ—R^{c})O]_{y}—Si—(R¹³)₃ Formula V
wherein p has a value of 6 to 16; x has a value of 6 to 100; and y has a value of 1 to 20 and the other moieties have the same definition as defined in Formula IV.

It should be understood that in both Formulas I and II shown above, that the siloxane-oxyalkylene copolymers of the present invention may, in alternate embodiments, take the form of endblocked polyethers in which the linking group R^{b}, the oxyalkylene segments, and the terminating radical R^{c} occupy positions bonded to the ends of the siloxane chain, rather than being bonded to a silicon atom in the siloxane chain. Thus, one or more of the R¹⁰, R¹¹, R¹² and R¹³ substituents which are attached to the two terminal silicon atoms at the end of the siloxane chain can be substituted with the segment —R—O—(C₂H₄O)ₚ—(C₃HO)ₛ—R^{c} or with the segment —R—O—(C₂H₄O)ₚ—R^{c}. In some instances, it may be desirable to provide the segment —R^{b}—O—(C₂H₄O)ₚ—(C₃H₆O)ₛ—R^{c} or the segment —R^{b}—O—(C₂H₄O)ₚ—R^{c} at locations which are in the siloxane chain as well as at locations at one or both of the siloxane chain ends.

Particular examples of suitable dimethicone copolyols are available either commercially or experimentally from a variety of suppliers including Dow Corning Corporation, Midland, Mich.; General Electric Company, Waterford, N.Y.; Witco Corp., Greenwich, Conn.; and Goldschmidt Chemical Corporation, Hopewell, Va. Examples of specific products include DOW CORNING® 5225C from Dow Corning which is a 10% dimethicone copolyol in cyclomethicone; DOW CORNING® 2-5185C which is a 45-49% dimethicone copolyol in cyclomethicone; SILWET L-7622 from Witco; ABIL EM97 from Goldschmidt which is a 85% dimethicone copolyol in D5 cyclomethicone; and various dimethicone copolyols available either commercially or in the literature.

It should also be noted that various concentrations of the dimethicone copolyols in cyclomethicone can be used. While a concentration of 10% in cyclomethicone is frequently seen commercially, other concentrations can be made by stripping off the cyclomethicone or adding additional cyclomethicone. The higher concentration materials such as DOW CORNING® 2-5185 material is of particular interest.

In one particular embodiment 0.5-50 weight % (particularly 10-30%) of a 10% silicone copolyol such as dimethicone copolyol in cyclomethicone mixture may be used, wherein the amount of mixture added is selected so that the level of silicone copolyol in the cosmetic composition is in the range of 0.05-5.0% (particularly 0.1-3.0%).

The antiperspirant actives that can be utilized according to the present invention are conventional aluminum and aluminum/zirconium salts, as well as aluminum/zirconium salts complexed with a neutral amino acid such as glycine (“gly”), as known in the art. See each of European Patent Application Number 512,770 A1 and PCT case WO 92/19221, the contents of each of which are incorporated herein by reference in their entirety, for disclosure of antiperspirant active materials. The antiperspirant active materials disclosed therein, including the acidic antiperspirant materials, can be incorporated in the compositions of the present invention. Suitable materials include (but are not limited to) aluminum chlorohydroxide, aluminum chloride, aluminum sesquichlorohydroxide, zirconyl hydroxychloride, and aluminum chlorohydrol-propylene glycol complex. These include, by way of example (and not of a limiting nature), aluminum chlorohydrate, aluminum chloride, aluminum sesquichlorohydrate, zirconyl hydroxychloride, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), and mixtures of any of the foregoing. The aluminum-containing materials can be commonly referred to as antiperspirant active aluminum salts. Generally, the foregoing metal antiperspirant active materials are antiperspirant active metal salts. In the embodiments which are antiperspirant compositions according to the present invention, such compositions need not include aluminum-containing metal salts, and can include other antiperspirant active materials, including other antiperspirant active metal salts. Generally, Category I active antiperspirant ingredients listed in the Food and Drug Administration's Monograph on antiperspirant drugs for over-the-counter human use can be used. In addition, any new drug, not listed in the Monograph, such as tin or titanium analogues of the aluminum salts listed above, aluminum nitratohydrate and its combination with zirconyl hydroxychlorides and nitrates, or aluminum-stannous chlorohydrates, can be incorporated as an antiperspirant active ingredient in antiperspirant compositions according to the present invention. Preferred antiperspirant actives that can be incorporated in the compositions of the present invention include the enhanced efficacy aluminum salts and the enhanced efficacy zirconium/aluminum salt-glycine materials, having enhanced efficacy due to improved molecular distribution, known in the art and discussed, for example, in PCT No. WO92/19221, the contents of which are incorporated by reference in their entirety herein.

Antiperspirant actives can be incorporated into compositions according to the present invention in amounts in the range of 0-10% (on an anhydrous solids basis), preferably 5-10%, by weight, of the total weight of the composition. The amount used will depend on the formulation of the composition. For example, at amounts in the lower end of the broader range (for example, 0.1-5%), the antiperspirant active material will not substantially reduce the flow of perspiration, but will reduce malodor, for example, by acting as a deodorant material, for example, by acting as an antimicrobial or complexing with the malodorous components of human perspiration.

Deodorant active materials can include lesser amounts of antiperspirant actives, such as in the range of 0.1-5%, as well as fragrances, and effective amounts of antimicrobial agents, for example, farnesol, bacteriostatic quaternary ammonium compounds (such as cetyl trimethyl-ammonium bromide, and cetyl pyridinium chloride), 2,4,4′-trichloro-2′-hydroxydiphenylether (Triclosan), N-(4-chlorophenyl)-N′-(3,4-dichlorophenyl)urea (Triclocarban), silver halides, octoxyglycerin (SENSIVA™ SC 50) and various zinc salts (for example, zinc ricinoleate) may also be included in formulations of the present invention. The bacteriostat can, illustratively, be included in the composition in an amount of 0.01-5.0% by weight, of the total weight of the composition. Triclosan or Triclocarban can, illustratively, be included in an amount of from 0.05% to about 5.0% by weight, of the total weight of the composition.

Non-volatile silicones may also be used in the formulations of this invention. Such nonvolatile silicones have a flash point greater than 100 degrees C. and a viscosity in the range of 6-1000 centistokes. Suitable non volatile silicones include linear organo-substituted polysiloxanes which are polymers of silicon/oxygen with a general structure:
(1) (R¹⁰)₃SiO(Si (R¹¹)₂O)ₓSi(R¹²)₃ where R¹⁰, R¹¹ and R¹² can be the same or different and are each independently selected from the group consisting of phenyl and C1-C60 alkyl; or
(2) HO(R¹⁴)₂SiO(Si(R¹⁵)₂O)ₓSi(R¹⁶)₂OH, where R¹⁴, R¹⁵ and R¹⁶ can be the same or different and are each independently selected from the group consisting of phenyl and C1-C60 alkyl.
   Specific examples include dimethicone, dimethiconol behenate, C₃₀₋₄₅ alkyl methicone, stearoxytrimethylsilane, phenyl trimethicone and stearyl dimethicone.

Emollients are a known class of materials in this art, imparting a soothing effect to the skin. These are ingredients that help to maintain the soft, smooth, and pliable appearance of the skin. Emollients are also known to reduce whitening on the skin and/or improve aesthetics. Examples of chemical classes from which suitable emollients can be found include:
(a) fats and oils which are the glyceryl esters of fatty acids, or triglycerides, normally found in animal and plant tissues, including those which have been hydrogenated to reduce or eliminate unsaturation. Also included are synthetically prepared esters of glycerin and fatty acids. Isolated and purified fatty acids can be esterified with glycerin to yield mono-, di-, and triglycerides. These are relatively pure fats which differ only slightly from the fats and oils found in nature. The general structure may be represented by Formula VI: 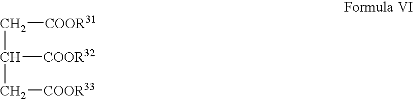
   wherein each of R³¹, R³², and R³³ may be the same or different and have a carbon chain length (saturated or unsaturated) of 7 to 25. Specific examples include peanut oil, sesame oil, avocado oil, coconut, cocoa butter, almond oil, safflower oil, corn oil, cotton seed oil, castor oil, hydrogenated castor oil, olive oil, jojoba oil, cod liver oil, palm oil, soybean oil, wheat germ oil, linseed oil, and sunflower seed oil;
(b) hydrocarbons which are a group of compounds containing only carbon and hydrogen. These are derived from petrochemicals. Their structures can vary widely and include aliphatic, alicyclic and aromatic compounds which have 7-40 carbons. Specific examples include paraffin, petrolatum, hydrogenated polyisobutene, and mineral oil;
(c) esters which chemically are the covalent compounds formed between acids and alcohols. Esters can be formed from almost all acids (carboxylic and inorganic) and any alcohol. Esters here are derived from carboxylic acids and an alcohol. The general structure would be R³⁴CO—OR³⁵. The total number of carbons for R³⁴ and R³⁵ together can vary from 7 to 40 and can be saturated or unsaturated, straight chained or branched or can include an aromatic structure. Specific examples include isopropyl myristate, isopropyl palmitate, isopropyl stearate, isopropyl isostearate, butyl stearate, octyl stearate, hexyl laurate, cetyl stearate, diisopropyl adipate, isodecyl oleate, diisopropyl sebacate, isostearyl lactate, C₁₂₋₁₅ alkyl benzoates, myreth-3 myristate, dioctyl malate, neopentyl glycol diheptanoate, neopentyl glycol dioctanoate, dipropylene glycol dibenzoate, C₁₂₋₁₅ alcohols lactate, isohexyl decanoate, isohexyl caprate, diethylene glycol dioctanoate, octyl isononanoate, isodecyl octanoate, diethylene glycol diisononanoate, isononyl isononanoate, isostearyl isostearate, behenyl behenate, C₁₂-15 alkyl fumarate, laureth-2 benzoate, propylene glycol isoceteth-3 acetate, propylene glycol ceteth-3 acetate, octyldodecyl myristate, cetyl ricinoleate, myristyl myristate (with a particular ester of interest being C12-15 alkyl benzoate);
(d) saturated and unsaturated fatty acids which are the carboxylic acids obtained by hydrolysis of animal or vegetable fats and oils. These have general structure R³⁶COOH with the R³⁶ group having a carbon chain length of 7-25 and R³⁶ can be straight chain or branched. Specific examples include lauric, myristic, palmitic, stearic, oleic, linoleic and behenic acid;
(e) saturated and unsaturated fatty alcohols (including guerbet alcohols) with general structure R³⁷COH where R³⁷ can be straight chain or branched and have a carbon chain length of 7 to 30. Specific examples include lauryl, myristyl, cetyl, isocetyl, stearyl, isostearyl, oleyl, ricinoleyl and erucyl alcohol;
(f) lanolin and its derivatives which are a complex esterified mixture of high molecular weight esters of (hydroxylated) fatty acids with aliphatic and alicyclic alcohols and sterols. General structures would include R³⁸CH₂—(OCH₂CH₂)ₙOH where R³⁸ represents the fatty groups derived from lanolin and n=5 to 75 or R³⁹CO—(OCH₂CH₂)ₙH where R³⁹CO— represents the fatty acids derived from lanolin and n=5 to 100. Specific examples include lanolin, lanolin oil, lanolin wax, lanolin alcohols, lanolin fatty acids, isopropyl lanolate, ethoxylated lanolin and acetylated lanolin alcohols;
(g) alkoxylated alcohols wherein the alcohol portion is selected from aliphatic alcohols having 2-18 and more particularly 4-18 carbons, and the alkylene portion is selected from the group consisting of ethylene oxide, and propylene oxide having a number of alkylene oxide units from 2-53 and, more particularly, from 2-15. Examples include cetyl glyceryl ether, isostearyl glyceryl ether, isostearyl glyceryl pentaerythrityl ether, laureth-5 butyl ether, oleyl glyceryl ether, PEG-4 ditallow ether, polyglyceryl-3 cetyl ether, polyglyceryl-4 lauryl ether, PPG-9 diglyceryl ether, and propylene glycol myristyl ether. More specific examples include PPG-14 butyl ether, PPG-53 butyl ether, laureth-5 butyl ether, and PEG-4 ditallow ether;
(h) ethers selected from the group consisting of dicapryl ether, dicetyl ether, dimethyl ether, distearyl ether, ethyl ether, isopropyl hydroxycetyl ether, methyl hexyl ether, and polyvinyl methyl ether;
(i) adipic acid blends selected from the group consisting of trimethyl pentanediol/adipic acid copolymer (LEXOREZ TL8 from Inolex, Philadelphia, Pa.), trimethyl pentanediol/adipic acid/isononanoic acid copolymer (LEXOREZ TC8), and adipic acid/diethylene glycol/glycerin crosspolymer (LEXOREZ 100); and

(j) mixtures and blends of two or more of the foregoing.

One particular group of emollients includes C12-15 alkyl benzoate (FINSOLV TN from Finetex Inc., Elmwood Park, N.J.), medium volatility dimethicone (especially 10-350 centistoke material and more especially 10-200 centistoke material), isopropyl myristate; and neopentyl glycol diheptanoate.

Particular examples of suitable emollients include members of the group consisting of Octyloxyglycerin (SENSIVA SC50 from Schulke Mayr, Nordstedt, Germany) (which can be used as an emollient as well as an antibacterial); ethoxylated alcohols such as steareth-2, nonoxynol-2, PPG-4-Ceteth-1; ethoxylated carboxylic acids such as PEG-4 dilaurate, PEG-2 oleate; glyceryl esters such as PEG-2 castor oil, polyglyceryl-3 oleate, glyceryl stearate; sorbitan derivatives such as sorbitan oleate; PPG-3 myristyl ether (such as WITCONOL APM from Goldschmidt); a dimethiconol (such as Dow Corning® DC 1501 dimethiconol); neopentyl glycol diheptanoate; PEG-8 laurate, isocetyl stearate; isostearyl isostearate; isostearyl palmitate; isostearyl alcohol; PPG-5-ceteth-20; PPG-10-cetyl ether; triethyl hexanoin; ethyl hexyl isostearate, glyceryl oleate, and isopropyl isostearate.

The emollient or emollient mixture or blend thereof incorporated in compositions according to the present invention can, illustratively, be included in amounts of 1-15%, and particularly 3-12% by weight of the total weight of the composition.

Baseline Absorption Test

A stick composition is made as described in Example 6, below. A second composition is made as a control except that no superabsorbent (“SA”) is used. Samples (2 grams in the form of shavings of the stick product) of each of these compositions are weighed into separate 16×100 mm Kimax disposable culture tubes. Water (2.0 g) is added to each of the tubes. The tubes are centrifuged for 5 minutes at 3000 rpm whereby the water, if not completely absorbed, settles at the bottom of the tube. The % water absorption is calculated as:

$$\frac{ \begin{matrix}( \text{height of the water in control} - \\ height of water in tube with SA )\end{matrix} }{\text{height of water in control}} \times 100$$

In performing the Baseline Absorption Test for the standard of this invention, the ratio of water:superabsorber is set as 20:1 by weight amounts. (Note that data is included below for 10:1 and 30:1 ratios, but the standard test to be used is using a 20:1 ratio).

The Baseline Absorption Test is important because not all superabsorbents will work in this invention. The compositions of this invention have a brutal environment from the standpoint of salt content, especially for antiperspirant products which contain about 15-22 weight % of an active salt such as an aluminum zirconium tetrachlorohydrex glycine material. In order to select an appropriate superabsorber which can maintain sufficient capacity in a high salt environment, it has been found that the Baseline Absorption Test is the best predictor of which superabsorbers will work. Other parameters such as particle size do not appear to show any consistent trends.

The compositions of this invention include sticks and soft solids. The compositions of the invention may range in clarity from opaque to white.

For deodorant stick products, the following general amounts of ingredients may be used:

Formulation A

(a) 5-25 weight % (particularly 8-20%) superabsorbent polymer as described above;
(b) 10-25 weight % of a gellant (for example, selected from the group consisting of silicone elastomer of the type described above (for example, KSG-15 from Shin-Etsu or DC 9040 from Dow Corning), stearyl alcohol, waxes (both low and/or high melting point waxes), hydrogenated castor oil, and low molecular weight polyethylene (such as a molecular weight of about 400 for example, Performalene-400);
(c) 40-70 weight % of a volatile silicone selected from the group consisting of a cyclomethicone (for example, one or more of D4, D5 or D6);
(d) 0-15 weight % of a non-volatile silicone which is a dimethicone having a viscosity in the range of 6-1000 centistokes;
(e) 2-10 weight % of an emollient selected from the group consisting of polyisobutene, and C12-15 alkyl benzoates (such as FINSOLV TN);
(f) 0-5 weight % (especially 1-3%) fragrance;
(g) 0-10 weight % (particularly 1-5%) surfactants (for example, PEG-8 distearate or PPG-3 myristyl ether);
(h) 0-5% antiperspirant active; and
(i) less than 2 weight % water.

For soft solid deodorant products, the following general amounts of ingredients may be used:

Formulation B

(a) 70-99.94 weight % silicone elastomer of the type described above (for example, KSG-15 or DC 9040);
(b) 0.01-30 weight % superabsorbent of the type described above;
(c) 0-5% antiperspirant active;
(d) 0-5 weight % fragrance; and
(e) less than 2 weight % water.

For antiperspirant stick products containing superabsorber, the following general amounts of ingredients may be used:

Formulation C

(a) 1-10 weight % (particularly 2-8%) superabsorbent polymer as described above;
(b) 10-25 weight % of a gellant (for example, selected from the group consisting of silicone elastomer of the type described above (for example, KSG-15 from Shin-Etsu or DC 9040 from Dow Corning), stearyl alcohol, waxes (both low and/or high melting point waxes), hydrogenated castor oil, and low molecular weight polyethylene (such as a molecular weight of about 400 for example, Performalene-400);
(c) 40-70 weight % of a volatile silicone selected from the group consisting of a cyclomethicone (for example, one or more of D4, D5 or D6);
(d) 0-15 weight % of a non-volatile silicone which is a dimethicone having a viscosity in the range of 6-1000 centistokes;
(e) 2-15 weight % of an emollient selected from the group consisting of polyisobutene, and C12-15 alkyl benzoates (such as FINSOLV TN);
(f) 0-5 weight % (especially 1-3%) fragrance;
(g) 0-10 weight % (particularly 1-5%) surfactants (for example, PEG-8 distearate or PPG-3 myristyl ether);
(h) 10-25% antiperspirant active; and
(i) less than 2 weight % water.

For soft solid antiperspirant products containing superabsorber, the following general amounts of ingredients may be used:

Formulation D

(a) 50-80 weight % silicone elastomer of the type described above (for example, KSG-15 or DC 9040);
(b) 0.01-10 weight % superabsorbent of the type described above;
(c) 10-25% antiperspirant active;
(d) 0-5 weight % fragrance; and
(e) less than 2 weight % water.

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights and degree of ethoxylation or propoxylation are averages. Temperatures are in degrees C. unless otherwise indicated. The amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7th ed. 1997).

### Examples 1 and 3

### Deodorant Stick Products

A stick product of about 400 grams can be made using the ingredients listed in Table A. The dimethicone (DC 200, 10 censtistokes from Dow Corning Corporation, Midland, Mich.) and C12-15 alkyl benzoate (FINSOLV TN, from Finetex Elmwood Park, N.J.) (and polyisobutene and PPG-3 myristyl ether for Example 3) are added to a suitable size first beaker and heated with stirring to 55-60 degrees C. The Japan wax substitute 525 (if used) is added and mixed until melted. The temperature is increased to 82-85 degrees C. and the low molecular weight polyethylene (Performalene-400 from Baker Petrolite) is added and mixed until melted. The mixture is then cooled to a temperature of about 80 degrees C. In a separate second beaker the silicone elastomer (KSG-15 from Shin-Etsu Silicones of America, Akron, Ohio) is added followed by the addition of the cyclomethicone (Cyclomethicone 345 from Dow Corning Corporation, Midland, Mich.). The mixture is stirred for about 5 minutes and then heated to a temperature of about 70 degrees C. The silicone elastomer/cyclomethicone mixture from the second beaker is then added to the first beaker with continuous stirring while maintaining the temperature at 78-80 degrees C. The superabsorbent material (HySorb™ 8100, BASF, N.C. ), ground to particle size less than 100 microns, and the antiperspirant active (active as described in Example 3), if used, are then added at this temperature and stirred for 10 minutes. The fragrance is added at the same 78-80 degrees C. temperature and stirred for 1 minute. The product is poured into suitable containers (size is approximately 3 cm (width at widest part of oval)×6 cm (length of base)×10 cm (height) with an ovoid shape) at 78-80 degrees C. and cooled for 15 minutes in a refrigerator at about 4 degrees C. and then at room temperature.

### Example 2

### Deodorant Stick Product

A stick product of about 400 grams may be made using the ingredients listed in Table A. The cyclomethicone and dimethicone are added to a suitable size beaker and heated to a temperature of about 70 degrees C. Stearyl alcohol is added with stirring at 70 degrees C. until it is melted. PEG-8 distearate is added with mixing while maintaining the temperature at 70 degrees C. until it is dissolved. The temperature of the mixture is then increased to about 80 degrees C. Hydrogenated castor oil is added with mixing at 80 degrees C. until it is completely dissolved. The mixture is cooled to about 75 degrees C., the superabsorbent material is added with stirring, and the temperature is maintained at 70-75 degrees C. for 15 minutes. The mixture is cooled to about 65 degrees C. and fragrance is added. The mixture is then cooled to about 58 degrees C. and then poured into appropriate containers as described in Example 1.

TABLE A

| Ingredients (weight %) | Ex. 1 | Ex. 2 | Ex. 3 |
|---|---|---|---|
| Superabsorbent polymer | 20 | 10 | 5 |
| (HySorb ™ 8100; particle | | | |
| size less than 100 microns) | | | |
| Dimethicone (10 cst) | 10 | 12 | — |
| C12-15 alkyl benzoate | 5 | — | 7 |
| Japan Wax Substitute 525 | 3 | — | — |
| Cyclomethicone 345 | 27.8 | 50 | 50.3 |
| Polyethylene (Performalene-400) | 8 | — | 10 |
| Silicone elastomer (KSG-15) | 25 | | 15 |
| Fragrance | 1.2 | 1.2 | 1.2 |
| Stearyl alcohol | — | 20 | — |
| Hydrogenated castor oil | — | 4 | — |
| PEG-8 distearate | — | 4 | — |
| PPG-3 myristyl ether | — | — | 4 |
| Polyisobutene 250 | — | — | 5 |
| Antiperspirant active (AZZ902) | | | 2.5 |
| Total | 100 | 100 | 100 |

### Example 4

### Soft Solid Deodorant Product

A soft solid product of about 400 grams may be made using the following ingredients. A silicone elastomer (97% of Dow 9040 from Dow Corning), superabsorbent polymer (2% of the same one used in Example 1) and fragrance (1%) are combined with mixing in a Hobart mixer at room temperature for about 15-20 minutes.

### Examples 5, 6 and 7

### Antiperspirant Stick Product with Superabsorber

A stick product of about 400 grams may be made using the ingredients listed in Table A. The cyclomethicone and C12-15 alkyl benzoate are added to a suitable size beaker and heated to a temperature of about 70 degrees C. Stearyl alcohol is added with stirring at 70 degrees C. until it is melted. PEG-8 distearate is added with mixing while maintaining the temperature at 70 degrees C. until it is dissolved. The temperature of the mixture is then increased to about 80 degrees C. Hydrogenated castor oil is added with mixing at 80 degrees C. until it is completely dissolved. The mixture is cooled to about 75 degrees C., the antiperspirant active and superabsorbent materials are added with stirring, and the temperature is maintained at 70-75 degrees C. 20 for 15 minutes. The mixture is cooled to about 65 degrees C. and fragrance is added.

The mixture is then cooled to about 58 degrees C. and then poured into appropriate containers as described in Example 1.

TABLE B

| Ingredients (weight %) | Ex. 5 | Ex. 6 | Ex. 7 |
|---|---|---|---|
| Superabsorbent polymer (HySorb ™ 8100; | 2.50 | 5.00 | 10.00 |
| particle size less than 100 microns) | | | |
| C12-15 alkyl benzoate | 12.00 | 12.00 | 12.00 |
| Cyclomethicone 345 | 34.30 | 35.80 | 37.80 |
| Stearyl alcohol | 20.00 | 16.00 | 16.00 |
| Hydrogenated castor oil | 4.00 | 4.00 | 4.00 |
| PEG-8 distearate | 4.00 | 4.00 | 4.00 |
| Antiperspirant active (Summit Z576) | 22.00 | 22.00 | 15.00 |
| Fragrance | 1.20 | 1.20 | 1.20 |
| Total | 100 | 100 | 100 |

### Water Absorption of Deodorants, Examples 1-4

In formulations containing zero or low levels of antiperspirant salts, i.e. at low ionic strength, (Examples 1-4), high water absorption capacity of the formulations were observed. This was shown through the following experiment. Samples (2.0 g) of the formulations from each of Examples 1-4 were weighed into 16×100 mm Kimax disposable culture tubes and 1.0 and 2.0 g of water were added to the formulations. The tubes were centrifuged for 5 minutes at 3000 rpm whereby the water, if not absorbed, settled at the bottom of the tubes. Examples 1-4 showed no residual water, indicating that all the water was absorbed in these formulations. Thus, when the antiperspirant active salt is low, water absorption by the superabsorbent is high.

### Water Absorption of Antiperspirants Containing Different Superabsorbents

The water absorption capacity of superabsorbent polymers are known to be affected by salts, such as sodium chloride or an antiperspirant active. Examples 6 and 8 (TABLE C) show two formulations, one containing a superabsorbent which is more salt tolerant (HySorb™ 8100, from BASF, Charlotte, N.C.) and the other containing a starch graft copolymer of poly (2-propenamide-co-2-propenoic acid, sodium salt) (“SGC”) and is not as salt tolerant. SGC stands for “starch graft copolymer”.

TABLE C

| Ingredients (weight %) | Ex. 6 | Ex. 8 | Ex. 9 |
|---|---|---|---|
| Superabsorbent polymer (HySorb ™ 8100; | 5.00 | | |
| particle size less than 100 microns) | | | |
| SGC | | 5.00 | |
| C12-15 alkyl benzoate | 12.00 | 12.00 | 12.00 |
| Cyclomethicone 345 | 35.80 | 35.80 | 36.80 |
| Stearyl alcohol | 16.00 | 16.00 | 20.00 |
| Hydrogenated castor oil | 4.00 | 4.00 | 4.00 |
| PEG-8 distearate | 4.00 | 4.00 | 4.00 |
| Antiperspirant active (Summit Z576) | 22.00 | 22.00 | 22.00 |
| Fragrance | 1.20 | 1.20 | 1.20 |
| Total | 100 | 100 | 100 |

Examples 6 and 8 were compared for their water absorption water capacity versus 5 Example 9 (no superabsorber) as control. Samples (2.0 g) of the formulations as shavings were weighed into 16×100 mm Kimax disposable culture tubes. Water in three different amounts (1.0, 2.0 and 3.0 g) were added to the formulations. This corresponds to water/superabsorber ratios of 10:1, 20:1 and 30: 1, respectively. The tubes were centrifuged for 5 minutes at 3000 rpm whereby the water, if not absorbed, settled at the bottom of the tubes. The height of the water was measured (in mm) and the results are tabulated in Table D.

TABLE D

| | | Ratio | Height of water | |
|---|---|---|---|---|
| Sam- | Water | of | in tube after | % Water |
| ple | added (g) | water:superabsorber | centrifugation (mm) | absorption |
| Ex 9 | 1.00 | — | 6.0 | |
| Ex 8 | 1.00 | 10 | 5.0 | 16.6 |
| Ex 6 | 1.00 | 10 | 0 | 100 |
| Ex 9 | 2.00 | — | 12.1 | |
| Ex 8 | 2.00 | 20 | 10.1 | 16.5 |
| Ex 6 | 2.00 | 20 | 4.0 | 67 |
| Ex 9 | 3.00 | — | 18.0 | |
| Ex 8 | 3.00 | 30 | 15.0 | 16.7 |
| Ex 6 | 3.00 | 30 | 8.5 | 53 |

The results clearly demonstrate that HySorb™ 8100 superabsorbent is significantly more effective in absorbing water in the presence of a ZAG than the SGC material. At a water/superabsorber ratio of 10:1, 100% water is absorbed from the formulation containing HySorb™ 8100 superabsorbent as opposed to only about 16.6% for the formulation containing the SGC material. At 20:1 water superabsorber ratio, about 67% of water is absorbed for the formula containing HySorb™ 8100 superabsorbent compared to 16.5% for the formulation containing SGC material. At 30:1 ratio, 53% of water is absorbed for the formulation containing HySorb™ 8100 superabsorbent compared to 16.7% for the formulation containing SGC material. Thus, at all three water/superabsorber ratios, the formulation containing HySorb™ 8100 superabsorbent performed more efficiently in absorbing water than the formulation containing SGC material. Taken together, the data indicate that the HySorb™ 8100 product absorbs water more effectively especially at high salt concentration, the concentration needed to claim antiperspirant efficacy. Therefore, it can be used in an antiperspirant product to boost the efficacy of the ZAG at levels up to 25 weight %.

### Examples 6-9 and 10-12

Comparison of water absorbency for different superabsorbers (all of which are polyacrylates) was done on the following polyacrylate, sodium salt samples as listed in TABLE F: (a) material with a mean particle size of 20-50 microns and a bulk density of 0.65 g/ml (SANFRESH ST-500 MPSA (obtained from Sanyo Chemical Industries, Japan)); (b) material with a mean particle size of 200-300 microns and a bulk density of 0.34-046 g/ml (AQUA KEEP J-550) and material with a mean particle size of 20-30 microns and a bulk density of 0.84-0.96 g/ml (AQUA KEEP 10SH-NF) (both obtained from Kobo Products, Inc., South Plainfield, N.J.). The basic formula was made by combining the ingredients as listed above or as listed in TABLE E using the technique described for Examples 5-7. For the evaluation, 2 grams of water were added to 2 grams of each of the formulas and the procedure described above for Examples 6, 8 and 9 was followed. The ratio of water:superabsorber=20:1. The resulting values of water height after centrifugation are in TABLE F and show the better performance of Examples 6, 10 and 11 as compared to Control (Example 9) and other superabsorbers that do not perform as well in a salt environment (Examples 8 and 12).

TABLE E

| Ingredient | Ex. 10 | Ex. 11 | Ex. 12 |
|---|---|---|---|
| Superabsorbent polymer (SANFRESH | 5.00 | | |
| ST-500 MPSA) | | | |
| Superabsorbent polymer (AQUA | | 5.00 | |
| KEEP J-550) | | | |
| Superabsorbent polymer (AQUA | | | 5.00 |
| KEEP 10SH-NF) | | | |
| C12-15 alkyl benzoate | 12.00 | 12.00 | 12.00 |
| Cyclomethicone 345 (Dow Corning) | 35.80 | 35.80 | 35.80 |
| Stearyl alcohol | 16.00 | 16.00 | 16.00 |
| Hydrogenated castor oil (MP 80) | 4.00 | 4.00 | 4.00 |
| PEG-8 distearate | 4.00 | 4.00 | 4.00 |
| Antiperspirant active (Summit Z-576) | 22.00 | 22.00 | 22.00 |
| Fragrance | 1.20 | 1.20 | 1.20 |
| Total | 100.00 | 100.00 | 100.00 |

TABLE F

| | | | Water height | |
|---|---|---|---|---|
| | | | after centri- | |
| | | Water/ | fugation | % Water |
| Example | Formula | superabsorber | (mm) | Absorption |
| Ex. 9 | Base | — | 12.1 | |
| Ex. 8 | Base + | 20 | 10.1 | 16.5 |
| | 5% SGC | | | |
| Ex. 6 | Base + 5% | 20 | 4 | 67 |
| | Superabsorber | | | |
| | HySorb ™ | | | |
| | 8100 | | | |
| Ex. 10 | Base + 5% | 20 | 7 | 42 |
| | SANFRESH | | | |
| | ST-500 MPSA | | | |
| Ex. 11 | Base + 5% | 20 | 5.5 | 54.5 |
| | AQUAKEEP | | | |
| | J-550 | | | |
| Ex. 12 | Base + 5% | 20 | 9 | 25.6 |
| | AQUAKEEP | | | |
| | 10SH-NF | | | |

### Examples 1B-13B

Examples 12B-6B can be made with the method described below using the ingredient listed in TABLE G, except that another fragrance component such as a fragrance oil encapsulated with corn starch (for example, off the shelf products, customized products and/or proprietary products, for example, material obtained from Noville, South Hackensack, N.J.) is also used and added such as adding this additional fragrance component after the addition of the fragrance oil at a temperature in the range of 60-63 C. Data for water absorbency with the Baseline Absorbency Test is listed in Table H.

An alternative procedure “A” can also be used:
1. Mix and heat C1 2-15 alkyl benzoate and castorwax to 82-85 degrees C.
2. Add stearyl alcohol—temperature can be lowered to 75 degrees C.
3. Add PEG-8 distearate—temperature can be lowered to 72 degrees C.
4. Slowly add cyclomethicone but do not let temp go below 69 degrees C.
5. Add antiperspirant salt (Al/Zr gly at 72 degrees C. (use dust mask).
6. Add superabsorber (use dust mask).
7. Add fragrance and encapsulated fragrance at 60-63 degrees C.
8. Pour sticks at a temperature of 57-58 degrees C.

9. Cool in refrigerator for at least 15 minutes.

TABLE G

| | Ex. 1B | | | | | |
|---|---|---|---|---|---|---|
| Ingredient | (control) | Ex. 2B | Ex. 3B | Ex. 4B | Ex. 5B | Ex. 6B |
| Superabsorbent polymer | 0 | 3.00 | 0 | 0 | 4.00 | 5.00 |
| (HySorb ™ CL-15) | | | | | | |
| Superabsorbent polymer (AQUA | 0 | 0 | 3.00 | 0 | 0 | 0 |
| KEEP J-550) | | | | | | |
| Superabsorbent polymer (AQUA | 0 | 0 | 0 | 3.00 | 0 | 0 |
| KEEP 10SH-NF) | | | | | | |
| C12-15 alkyl benzoate | 12.00 | 12.00 | 12.00 | 12.00 | 12.00 | 12.00 |
| Cyclomethicone 345 (Dow | 35.50 | 32.50 | 32.50 | 32.50 | 31.50 | 30.50 |
| Corning) | | | | | | |
| Stearyl alcohol | 20.00 | 20.00 | 20.00 | 20.00 | 20.00 | 20.00 |
| Hydrogenated castor oil (MP 80) | 4.00 | 4.00 | 4.00 | 4.00 | 4.00 | 4.00 |
| PEG-8 distearate | 4.00 | 4.00 | 4.00 | 4.00 | 4.00 | 4.00 |
| Antiperspirant active | 22.00 | 22.00 | 22.00 | 22.00 | 22.00 | 22.00 |
| (Summit Z-576) | | | | | | |
| Fragrance | 1.00 | 1.00 | 1.00 | 1.00 | 1.00 | 1.00 |
| Fragrance #2 encapsulated | 1.50 | 1.50 | 1.50 | 1.50 | 1.50 | 1.50 |
| Total | 100.00 | 100.00 | 100.00 | 100.00 | 100.00 | 100.00 |

TABLE H

| | | | Water height | |
|---|---|---|---|---|
| | | | after | |
| | Water | Water/ | centrifugation | % Water |
| Example | added | superabsorber | (mm) | absorption |
| Ex. 1B | 1.2 | — | 6.3 | — |
| Ex. 2B | 1.2 | 20 | 5.2 | 17 |
| Ex. 3B | 1.2 | 20 | 3.7 | 41 |
| Ex. 4B | 1.2 | 20 | 5.4 | 13 |
| Ex. 1B | 1.6 | — | 9.7 | — |
| Ex. 5B | 1.6 | 20 | 7.4 | 24 |
| Ex. 1B | 2.0 | — | 12.7 | — |
| Ex. 6B | 2.0 | 20 | 9.0 | 29 |

### Examples 1C and 7B-9B

Examples 1C, 7B-9B can be made with the method described above for Examples 1B-6B, using the ingredients listed in TABLE I. Data for water absorbency with the Baseline Absorbency Test is listed in Table J.

TABLE I

| | Ex. 1C | | | |
|---|---|---|---|---|
| Ingredient | (control) | Ex. 7B | Ex. 8B | Ex. 9B |
| Superabsorbent polymer | 0 | 5.00 | 5.00 | 5.00 |
| (HYSORB (TM) CL-15) | | | | |
| C12-15 alkyl benzoate | 12.00 | 12.00 | 12.00 | 12.00 |
| Cyclomethicone 345 (Dow | 35.50 | 30.50 | 37.50 | 47.50 |
| Corning) | | | | |
| Stearyl alcohol | 20.00 | 20.00 | 20.00 | 20.00 |
| Hydrogenated castor oil (MP 80) | 4.00 | 4.00 | 4.00 | 4.00 |
| PEG-8 distearate | 4.00 | 4.00 | 4.00 | 4.00 |
| Antiperspirant active | 22.00 | 22.00 | 15.00 | 5.00 |
| (Summit Z-576) | | | | |
| Fragrance | 1.00 | 1.00 | 1.00 | 1.00 |
| Fragrance #2 encapsulated | 1.50 | 1.50 | 1.50 | 1.50 |
| Total | 100.00 | 100.00 | 100.00 | 100.00 |

TABLE J

| | | | Water height | |
|---|---|---|---|---|
| | | | after | |
| | Water | Water/ | centrifugation | % Water |
| Example | added | superabsorber | (mm) | absorption |
| Ex. 1C | 2.0 | — | 12.5 | — |
| Ex. 7B | 2.0 | 20.0 | 8.1 | 35 |
| Ex. 8B | 2.0 | 20.0 | 4.5 | 64 |
| Ex. 9B | 2.0 | 20.0 | 0.0 | 100 |

Thus, Example 2B could be modified to achieve the 25% required water absorption by lowering the antiperspirant active to 15% and/or raising the superabsorbent to 5%. Example SB could be modified to achieve the 25% required water absorption by lowering the amount of antiperspirant active to 15% and/or increasing the amount of superabsorbent to 5%.

Examples 1D and 11B-13B

Examples 1D, 11B-13B can be made with the method described above for Examples 1B-9B, using the ingredients listed in TABLE K except that talc can be added, for example, at a temperature of 72 degrees C. before the addition of the Al/Zr salt. The behenyl alcohol is added after the stearyl alcohol is added, again, for example, at a temperature of 72 degrees C. Mineral oil such as white mineral oil can be added with the C12-15 alkyl benzoate and heated to a temperature in the range of 82-85 degrees C. Data for water absorbency with the Baseline Absorbency Test is listed in Table L.

TABLE K

| | Ex. 1D | | | |
|---|---|---|---|---|
| Ingredient | (control) | Ex. 11B | Ex. 12B | Ex. 13B |
| Superabsorbent polymer | 0 | 3.00 | 4.00 | 5.00 |
| (HySorb ™ CL-15) | | | | |
| C12-15 alkyl benzoate | 12.00 | 12.00 | 12.00 | 12.00 |
| Cyclomethicone 345 (Dow | 34.00 | 31.00 | 30.00 | 29.00 |
| Corning) | | | | |
| Stearyl alcohol | 18.35 | 18.35 | 18.35 | 18.35 |
| Docosan-1-ol (behenyl alcohol) | 0.15 | 0.15 | 0.15 | 0.15 |
| Hydrogenated castor oil (MP 80) | 5.50 | 5.50 | 5.50 | 5.50 |
| PEG-8 distearate | 4.00 | 4.00 | 4.00 | 4.00 |
| Antiperspirant active (Summit | 20.00 | 20.00 | 20.00 | 20.00 |
| Z-576) | | | | |
| Talc | 2.00 | 2.00 | 2.00 | 2.00 |
| White mineral oil | 2.00 | 2.00 | 2.00 | 2.00 |
| Fragrance | 1.00 | 1.00 | 1.00 | 1.00 |
| Fragrance #2 encapsulated | 1.00 | 1.00 | 1.00 | 1.00 |
| Total | 100.00 | 100.00 | 100.00 | 100.00 |

TABLE L

| | | | Water height | |
|---|---|---|---|---|
| | | | after | |
| | Water | Water/ | centrifugation | % Water |
| Example | added | superabsorber | (mm) | absorption |
| Ex. 1D | 1.2 | — | 6.1 | — |
| Ex. 11B | 1.2 | 20.0 | 5.1 | 16 |
| Ex. 1D | 1.6 | | 7.5 | — |
| Ex. 12B | 1.6 | 20.0 | 6.2 | 17 |
| Ex. 1D | 2.0 | | 10.5 | — |
| Ex. 13B | 2.0 | 20.0 | 7.1 | 32 |

It should be noted that Example 11B and Example 12B could be altered to obtain a minimum 25 weight % water absorption value by raising the amount of superabsorber to 5 weight % and/or lowering the amount of antiperspirant salt to 10 weight %.

1. A stick or soft solid suspension product comprising:
(a) 0.01-20 weight % of a polyacrylate superabsorbent polymer (sodium salt), with a salt or ionic strength tolerance under a Baseline Absorption Test sufficient to give at least 25 weight % water absorption;
(b) 10-88 weight % of a volatile silicone having a flash point of 100 degrees C. or less; and
(c) a gelling agent selected from the group consisting of 5-30 weight % stearyl alcohol; 0.1-20 weight % waxes; 0.1-10 weight % (on an actives basis) silicone elastomer; 0.1-3 weight % siliconized polyamides; 0.1-20 weight % low molecular weight polyethylene having a molecular weight in the range of 400-1000 and combinations of the foregoing.

2. A product according to claim 1 further comprising one or more ingredients selected from the group consisting of:
(d) 0-5 weight % of a surfactant with a hydrophilic/lipophilic balance in the range of 3-13;
(e) 0-25 weight % of an antiperspirant active or. an effective amount of a deodorizing agent which is not an antiperspirant active;
(f) 0-20 weight % of a nonvolatile silicone having a flash point greater than 100 degrees C.; and
(g) 0-20 weight % of an emollient;
provided that the water content is ≦2 weight % and the ratio of superabsorber to active salt is in the range of 0.13-4:1.

3. A product according to claim 1 comprising 0.1-10% of the superabsorbent polymer.

4. A product according to claim 1 comprising 0.5-5% of the superabsorbent polymer.

5. A product according to claim 1 wherein the volatile silicone is a D4-D6 cyclomethicone.

6. A product according to claim 1 comprising one or both of D5 and D6 cyclomethicones as the volatile silicone.

7. A product according to claim 1 wherein the wax is Japan wax or a hydrogenated castor oil.

8. A product according to claim 1 wherein the surfactant comprises about from 0.05-50 weight % of a silicone copolyol at a concentration of 10% in cyclomethicone, or an equivalent amount of silicone copolyol using a different dilution factor.

9. A product according to any one of claims 1-8 further comprising 5-10% of an antiperspirant active.

10. A product according to claim 1 comprising 5-10% of the nonvolatile silicone.

11. A product according to claim 1 comprising 2-12% of the emollient.

12. A product according to claim 1 wherein the emollient is selected from the group consisting of C12-15 alkyl benzoate, PPG-3-myristyl ether, and polyisobutene 250 and comprises 2-12 weight % of the product.

13. A cosmetic product according to claim 1 further comprising an effective amount of a deodorizing agent which is not an antiperspirant active.

14. A cosmetic product according to claim 1 comprising 5-30 weight % stearyl alcohol as the gelling agent.

15. A cosmetic product according to claim 1 comprising 0.1-20 weight % waxes selected from the group consisting of Japan wax, hydrogenated castor oil with a melting point in the range of 50-90 degrees C. and mixtures thereof.

16. A cosmetic product according to claim 1 comprising a silicone elastomer as the gelling agent.

17. A product according to claim 1 comprising a low molecular weight polyethylene having a molecular weight in the range of 400-1000 as the gelling agent.

18. A product according to claim 1 comprising as the gelling agent a siliconized polyamide of Formula IIIA: 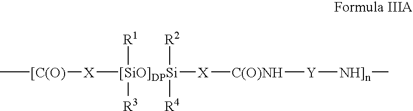
where:
(1) DP is a number in the range of 10-40;
(2) n is a number selected from the group consisting of 1-500;
(3) X is a linear or branched chain alkylene having 1-30 carbons;
(4) Y is selected from the group consisting of linear and branched chain alkylenes having 1-40 carbons, wherein:
(A) the alkylene group may optionally and additionally contain in the alkylene portion at least one of the members of a group consisting of (i) 1-3 amide linkages; (ii) C5 or C6 cycloalkane (as a cycloalkylene linkage); and (iii) phenylene optionally substituted by 1-3 members selected independently from the group consisting of C1-C3 alkyls; and
(B) the alkylene group itself may optionally be substituted by at least one member selected from the group consisting of (i) hydroxy; (ii) C3-C8 cycloalkane; (iii) 1-3 members selected independently from the group consisting of C1-C3 alkyls; phenyl optionally substituted by 1-3 members selected independently from the group consisting of C1-C3 alkyls; (iv) C1-C3 alkyl hydroxy; and (v) C1-C6 alkyl amine; or y=Z² where 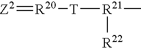
wherein each of R 20, R21 and R²² are independently selected from the group consisting of linear and branched C1-C10 alkylenes; and T is selected from the group consisting of (i) a trivalent atom selected from N, P and Al; and (ii) —CR, where R is selected from the group consisting of hydrogen, methyl, ethyl, propyl, isopropyl, a siloxane chain, and phenyl, wherein the phenyl may optionally be substituted by 1-3 members from the group consisting of methyl and ethyl; and
(5) each of R¹-R⁴ is independently selected from the group consisting of methyl, ethyl, propyl, isopropyl, a siloxane chain, and phenyl, wherein the phenyl may optionally be substituted by 1-3 members from the group consisting of methyl and ethyl; wherein the polyamide of Formula IIIA has:
(i) a silicone portion in the acid side of the polyamide;
(ii) a degree of polymerization in the range of 10-40;
(iii) an average molecular weight of at least 50,000 daltons with at least 95% of the polyamide having a molecular weight greater than 10,000 daltons; and
(iv) a polydispersity of less than 20.

19. A product according to any one of claims 1-8 and 10-18 wherein the ratio of superabsorbent polymer to active salt is in the range of 0.18-4:1.

20. A product according to any one of claims 1-8 and 10-18 wherein the superabsorbent polymer has a particle size not exceeding 100 microns.

21. A product according to any one of claims 1-8 and 10-18 wherein the superabsorbent polymer has a particle size ≦75 microns.

22. A product according to any one of claims 1-8 and 10-18 wherein the superabsorbent polymer has a particle size in the range of 6-65 microns with no more than 10% of the particles having a size less than 6 microns.
